(19) United States
(12) Patent Application Publication
(10) Pub. No.: US 2014/0296087 A1
(43) Pub. Date: Oct. 2, 2014

(54) NUCLEIC ACID BINDING OLIGONUCLEOTIDES

(71) Applicant: President and Fellows of Harvard College, Cambridge, MA (US)

(72) Inventors: Gregory L. Verdine, Newton, MA (US); Webster Santos, Somerville, MA (US)

(73) Assignee: President and Fellows of Harvard College, Cambridge, MA (US)

(21) Appl. No.: 14/063,233

(22) Filed: Oct. 25, 2013

Related U.S. Application Data
(63) Continuation of application No. 13/770,908, filed on Feb. 19, 2013, now abandoned.
Application No. 13/770,908 is a continuation of application No. 11/904,612, filed on Sep. 26, 2007, now abandoned.
Application No. 11/904,612 is a continuation of application No. 10/977,990, filed on Oct. 29, 2004, now abandoned.
(60) Provisional application No. 60/516,243, filed on Oct. 31, 2003.

(51) Int. Cl. C12Q 1/68

(52) U.S. Cl. CPC C12Q 1/6876
USPC 506/9; 506/16; 506/30

(57) ABSTRACT

The present application pertains to products and methods related to the ability of short nucleotide oligomers to bind the tertiary or globular structure of nucleic acids. This application discloses libraries of short oligomers and methods for using these libraries.

### BACKGROUND

Libraries and arrays of oligonucleotides have become integral tools in a wide range of fields from basic research to the identification of drug targets and drug discovery. A large industry has developed around the production of oligonucleotide libraries and arrays. One common use of these oligonucleotide libraries and arrays is for the detection of transcripts by detecting hybridization between complementary probe and nucleic acid sequences.

### SUMMARY

The present application discloses products and methods related to the ability of short nucleotide oligomers to bind the tertiary or globular structure of nucleic acids. This application discloses libraries of short oligomers and methods for using these libraries, e.g., for the purpose of identifying short oligomers that interact with nucleic acid targets in an interaction that includes at least one non-canonical interaction. As used in this application, non-canonical binding refers to binding forces that are independent of Watson-Crick, Hoogstein base-pairing rules.

### Libraries and Arrays

One embodiment of the present invention is a library of short nucleic acid oligomers containing oligomers that are 15 nucleotides in length or shorter (i.e. 15mers). Such a library may, for example, contain 14mers, 13mers, 12mers, 11mers, 10mers, 9mers, 8mers, 7mers, 6mers, 5mers, 4mers, or 3mers, or combinations thereof. Since shorter oligomers can effectively bind target nucleic acids, at least partially independently of base-pairing considerations, novel libraries that include short oligomers can be used to identify molecules that specifically recognize a target.

In another embodiment, the library of the present invention contains some fraction that is any percentage that is any percentage between 10, 20, 50 and less than 100%, inclusive, of all possible permutations of an oligomer of a particular length, where that particular length is a 15mer or shorter. A library of all possible permutations in this embodiment refers to a library containing every possible sequence arrangement of the four standard nucleotide monomers in an oligomer of a particular length (e.g., for a 6 mer, the library would include 4⁶=4096 unique species). For example, the library can contain some fraction that is any percentage between 50 and less than 100%, inclusive, of all possible DNA 5mer sequence permutations, where at each of its five positions, typically adjacent positions, the oligomer sequence contains either an Adenine (A), a Guanine (G), a Thymine (T) or a Cytosine (C). In another example the library contains some fraction that is any percentage between 50 and less than 100%, inclusive, of all possible RNA 4mer permutations, where at each of its four positions the oligomer contains either an Adenine (A), a Guanine (G), a Uridine (U), or a Cytosine (C). In other embodiments, the library includes all (or a substantial fraction thereof) permutations of any set of selected monomers (e.g., three standard nucleotide monomers, or a set that includes at least one non-standard nucleotide monomer). In one embodiment of a library that includes less than all possible permutations, the library omits one or more simple sequences, e.g., homopolymers (such as AAAAA), or other low complexity sequences (e.g., a sequence that includes only two types of nucleotides, or a sequence that includes only one change in nucleotide type (such as AATTT). In one embodiment of a library that includes less than all possible permutations, the library omits one or more sequences identified as promiscuous (e.g., binding to numerous targets without specificity) or binding to a common non-target structure, or one or more sequences that can form a stable intra-molecular interaction, e.g., a hairpin, or that would preferentially form a homoduplex (e.g., a palindromic sequence).

Typically the permuted positions are contiguous or refer to each possible position within the oligomer. However, in one embodiment, the permuted positions are a subset of the nucleotide positions within the oligomer. For example, each oligomer can include a common invariant region at one or both termini or internally.

Additional libraries of DNA, RNA and nucleic acid analog oligomers will be readily apparent to those of skill in the art upon reading this disclosure. It is important to note that in one embodiment, this invention includes the inclusion of one or more nucleotide analogs instead of a DNA or RNA nucleotide within the library's oligomers. In such a case the library contains some fraction that is any percentage between 50 and 100%, inclusive, of every sequence permutation of different nucleotides and nucleotide analogs used in the library, for an oligomer of a particular length. Alternatively, the oligomers in the library may contain entirely nucleotide analogs, in which case the library contains some fraction that is any percentage between 50 and 100%, inclusive, of every sequence permutation of nucleotide analogs used in the library, for an oligomer of a particular length. Oligomers in this library will be attached to a reporter tag or a support that enables identification and/or retrieval and/or separation of the oligomers that bind a particular target.

In another embodiment the library contains every possible permutation of a short, oligomer of a particular length, where that particular length can be from a 2mer to a 15mer, inclusive. In this embodiment, a library with every possible permutation refers to a library that contains at least one example of every unique sequence order that can be assembled using one or more of the four standard nucleotide base residues attached to a nucleic acid backbone in an oligomer of a particular length. For example, a DNA 5mer library, in this embodiment, contains at least one instance of every possible DNA 5mer sequence permutation, where at each of its five positions the oligomer sequence contains either an Adenine (A), a Guanine (G), a Thymine (T) or a Cytosine (C). Thus, the library includes 4⁵=1024 unique sequences.

In another embodiment the library contains exactly one instance of every unique sequence order that can be assembled using one or more of the four standard nucleotide base residues attached to a nucleic acid backbone in an oligomer of a particular length. For example, a DNA 5mer library, in this embodiment, contains exactly one instance of every possible DNA 5mer sequence permutation, where at each of its five positions the oligomer sequence contains either an Adenine (A), a Guanine (G), a Thymine (T) or a Cytosine (C). Thus, the library contains precisely 4⁵=1024 unique sequences.

Additional libraries of DNA, RNA and nucleic acid analog oligomers will be readily apparent to those of skill in the art upon reading this disclosure. In one embodiment, one or more nucleotide analogs are used instead of a DNA or RNA nucleotide in at least one, some, or all of the library's oligomers. One exemplary library contains every sequence permutation of different nucleotide(s) and nucleotide analog(s) used in the library, for an oligomer of a particular length. In another exemplary library, the library contains oligomers composed entirely of nucleotide analogs, in which case the library contains at least one instance of every sequence permutation of the nucleotide analogs used in the library, for an oligomer of a particular length. The oligomers of this library may contain sequence permutations of 2, 3, 4, 5 or 6 different nucleotide analogs. Oligomers in this library can be attached to a reported tag on a support that enables identification and/or retrieval and/or separation of the oligomers that bind a particular target.

In one embodiment, a library contains different oligomer types. Thus, for example, a library could contain one or more RNA oligomers, and the rest of the library could consist of DNA oligomers and/or nucleic acid analog oligomers. In another example, a library containing every sequence permutation of a DNA oligomer of a particular length except for one or more DNA oligomer permutations, would still be an embodiment of this invention if the library also contains RNA oligomers, or nucleic acid analog oligomers, whose base, or base analog, sequence is what persons of ordinary skill in the art of oligomer synthesis would recognize as a RNA base, or base analog, sequence that is equivalent to the missing DNA sequence permutations.

In a preferred set of embodiments, this invention features an array containing a plurality of short oligomers. The arrays of this invention can be a substrate upon which members of a library described herein are disposed. For example, the substrate can include an address for every oligomer in the library. One preferred embodiment features an array of short oligomers of a particular length, where that length is 15 nucleotides or nucleotide analogs or shorter. The array of this embodiment may contain a plurality of 15mers, 14mers, 13mers, 12mers, 11mers, 10mers, 9mers, 8mers, 7mers, 6mers, 5mers, 4mers, 3mers or 2mers. In another example, the substrate includes addresses that each contain a subset (e.g., a pool) of library members.

In another preferred embodiment, the array contains some fraction that is any percentage between 50 and less than 100%, inclusive, of all possible permutations of an oligomer of a particular length, where that particular length is a 15mer or shorter. An array of all possible permutations in this embodiment refers to an array that contains at least one instance of every sequence arrangement of the four standard nucleotide monomers that is possible in an oligomer of a particular length. In a different example, the array can contain some fraction that is any percentage between 50 and less than 100%, inclusive, of all possible DNA 5mer sequence permutations, where every oligomer permutation contains at each of its five positions the oligomer sequence contains either an Adenine (A), a Guanine (G), a Thymine (T) or a Cytosine (C). Another example is an array that contains some fraction that is any percentage between 50 and less than 100%, inclusive, of all possible RNA 4mer permutations, where at each of its four positions are oligomer contains either an Adenine (A), a Guanine (G), a Uridine (U), or a Cytosine (C).

Arrays of DNA, RNA and nucleic acid analog oligomers will be readily apparent to those of skill in the art upon reading this disclosure. It is important to note that this invention contemplates the inclusion of one or more nucleotide analogs instead of a DNA or RNA nucleotide within the array's oligomers. In such a case the array contains between 50 and 100%, inclusive, of every sequence permutation of different nucleotides and nucleotide analogs used in the array, for an oligomer of a particular length. Alternatively, the oligomers in the array may contain entirely nucleotide analogs, in which case the library contains between 50 and 100%, inclusive, of every sequence permutation of nucleotide analogs used in the array, for an oligomer of a particular length, where the length is a 15mer or shorter. Oligomers in these arrays may contain 1, 2, 3, 4, 5 or 6 different nucleotide analogs in combination with none, 1, 2, 3, 4, of 5 nucleotides.

In another embodiment, the array contains every possible permutation of a short, 15mer or less, oligomer of a particular length. In this embodiment, an array with every possible permutation refers to a library that contains at least one example of every unique sequence order of the four standard base residues attached to a nucleic acid backbone in an oligomer of a particular length. For example, a DNA 5mer array, in this embodiment, contains at least one instance of every possible DNA 5mer sequence permutations, where at each of its five positions the oligomer sequence contains either an Adenine (A), a Guanine (G), a Thymine (T) or a Cytosine (C).

Arrays of DNA, RNA and nucleic acid analog oligomers will be readily apparent to those of skill in the art upon reading this disclosure. It is important to note that this invention contemplates the use of one or more nucleotide analogs instead of a DNA or RNA nucleotide within the array's oligomers. In such a case the array contains every sequence permutation of different nucleotides and nucleotide analogs used in the array, for an oligomer of a particular length, where that length is a 15mer or shorter. Alternatively, the oligomers in the array may contain only nucleotide analogs, in which case the library contains every sequence permutation of those nucleotide analogs used in the array, for an oligomer of a particular length, where the length is a 15mer or shorter. Oligomers in these arrays may contain 1, 2, 3, 4, 5 or 6 different nucleotide analogs in combination with none, 1, 2, 3, 4, 5, or 6 nucleotides.

The present invention includes libraries that contain different oligomer types. A library does not fall outside the scope of the present invention merely because one or more oligomers in the library is of a different type as other oligomers in the library. Thus, for example, a library meeting all the other limitations of the present invention could contain one or more RNA oligomers, and the rest of the library could consist of DNA oligomers and/or nucleic acid analog oligomers. In another example, a library containing every sequence permutation of a DNA oligomer of a particular length except for one or more DNA oligomer permutations, would still be an embodiment of this invention if the library also contains RNA oligomers, or nucleotide analog oligomers, whose base, or base analog, sequence consists of what persons of ordinary skill in the art of oligomer synthesis would recognize as the RNA base or base analog sequence equivalent to the missing DNA sequence permutations.

Another set of embodiments of the present invention features any of the libraries or arrays described above, in which the oligomers of the library or array are stably associated with, or conjugated to a protein carrier molecule. For example, the Oligomer may be conjugated to the protein carrier and the protein carrier may be fixed to the support of a short oligomer library. In another example, the oligomer is conjugated to the protein carrier which is attached to the substrate of an array.

In one set of embodiments of the present invention, oligomers are attached to the support of any one of the libraries described herein by a spacer. In another set of embodiments of the present invention, oligomers are attached to the substrate of any one of the arrays described herein by a spacer. The spacer of either set of embodiments may be a nucleic acid or a non-nucleic acid.

The present invention includes embodiments Wherein any one of the libraries or arrays described herein further contains at least one oligomer bound to a target nucleic acid molecule that has some tertiary or globular structure. The target nucleic acid molecule may further be labeled. In preferred embodiments, the target nucleic acid molecule is radiolabeled, labeled with fluorescent reporter, or attached to a moiety that is suitable for secondary labeling.

Target molecules include DNA or RNA molecules with some tertiary or globular structure. Preferred embodiments feature RNA molecules with tertiary or globular structure. For example, a target RNA can include a mRNA, a rRNA, a tRNA, a non-protein coding RNA, a small RNA, a mRNA, a sRNA, a ribozyme, a spliceosome, a telomerase, a signal recognition particle, an untranslated 5′ or 3′ region within 100 nucleotides of an ATG start codon, a ribosome pause site, a rare codon, or an internal ribosome entry site (IRES). In one example, a target mRNA can include the RNA transcript of one of the following genes: an oncogene, a tumor suppressor gene, a cell adhesion molecule gene, or a telomerase. More specific examples include the mRNA from any one of the following: a translation initiation factor, e.g. eIF4G or DAP5; a transcription factor, e.g., c-myc, NF-B repressing factor (NRF); a growth factor e.g., Vascular endothelial growth factor (VEGF), Fibroblast growth factor 2 (FGF-2), Platelet-derived growth factor B (PDGF-B); a homeotic gene e.g., Antennapedia; a Survival Protein e.g., X-linked inhibitor of apoptosis (XIAP), Apaf-1; or BiP.

In another example, a target RNA may represent at least a portion of a viral nucleic acid sequence that is from one of the following: a RNA virus, a retrovirus, a dsRNA virus, a (+)sense RNA virus, a (−) RNA virus, a viroid, a satellite RNA, a prion encoding gene. Examples of viruses whose viral genomic sequence can be used as target RNAs include: Hepatitis C virus, Human Immnuodeficiency virus, Herpes virus, Kaposi's sarcoma-associated herpesvirus, Coronavirus, Bovine Coronavirus, Bovine viral diarrhea virus, GB virus-B, GB virus-C, Classic swine fever virus, foot-and-mouth disease virus, Friend murine leukemia virus, Moloney murine leukemia virus, Rous' sarcoma virus, Harvey sarcoma virus, Rhopalosiphum padi virus, Cricket paralysis virus, poliovirus, rhinovirus, encephalomyocarditis virus, and hepatitis A virus, or Plautia stali intestine virus (PSIV).

Target RNAs can be produced recombinantly in a host cell, by in vitro transcription, by in vitro chemical synthesis, by automated chemical synthesis, or target RNAs may be harvested from cells that produce the target RNA endogenously or from cells that have been infected with a virus.

### Apparatus

In another embodiment the invention features an apparatus that contains any of the novel libraries or arrays described above and a detector suitable for detecting an interaction between a target nucleic acid and an oligomer in the library or array. In one aspect of the invention, an oligomer in the library or array is associated with a target nucleic acid.

In a preferred embodiment, the detector of the apparatus is a scanner, for example a scanner capable of detecting fluorescence and/or radiolabels. Alternate embodiments have detectors that are microscopes. In a highly preferred embodiment, the apparatus includes a digital storage device that is suitable for storing information generated by the detector of the apparatus. For example, in the most preferred embodiment the detector generates information that is stored in a digital storage device it such a way that the information can be used (as the information is generated or at a later time) to determine the address and/or the oligomer to which a target nucleic acid has bound.

### Methods of Manufacturing a Library or Array

This invention also includes methods of manufacturing the novel libraries or arrays of the present invention. In one embodiment, the method includes providing a plurality of short oligomers of a particular length, where that length is a 15mer or shorter. The oligomers are attached to a reporter tag or a support that enables identification and/or retrieval and/or separation of the oligomers that bind a particular target nucleic acid molecule. In a preferred embodiment, the plurality of oligomers attached to a reporter tag or support constitutes any one of the libraries described above, e.g. a library that contains some fraction that is a percentage between 50 and 100%, inclusive, of every sequence permutation of nucleotides and/or nucleotide analogs used in the library, for an oligomer of a particular length. In another example, the library contains every possible sequence arrangement of the four standard nucleotide monomers in an oligomer of a particular length. In yet another example, the library contains every possible sequence arrangement of nucleotides and/or nucleotide analogs used in the library, for an oligomer of a particular length.

In a preferred embodiment, the invention includes a method for manufacturing the novel arrays of short oligomers that are disclosed above. The method includes providing a substrate that contains a plurality of addresses and attaching or synthesizing at least one short oligomer at each address, for some number of addresses on the substrate. The resulting array contains a plurality of oligomers of a particular length, where that length is between a 2mer and a 15mer, inclusive. In one embodiment, the method includes providing an address on the substrate for some fraction that is a percentage between 50 and less than 100%, inclusive, of all possible base sequence permutations of an oligomer of a particular length, where that particular length is a 5mer or shorter. Examples of this method include providing an address for some fraction between 50 and less than 100% of every sequence arrangement that can be assembled using the four standard nucleotide monomers in an oligomer of a particular length. In another example, one or more nucleotide analogs instead of a DNA or RNA nucleotide are included in the oligomers provided on the substrate, so that the method provides an address for some fraction that is a percentage between 50 and less than 100% of every possible sequence arrangement of the nucleotides and or nucleotide analogs used in the method. Oligomers used in this method may contain 1, 2, 3, 4, 5 or 6 different nucleotide analogs in combination with none, 1, 2, 3, 4, or 5 nucleotides.

In another embodiment, the method includes providing a substrate that contains a plurality of addresses, attaching or synthesizing at least one oligomer at each address for some number of addresses, such that at least one example of every unique sequence order that can be assembled in an oligomer of a particular length, where that length is a 15mer or shorter, using the nucleotides and/or nucleotide analogs is present on the substrate. For example, a method of manufacturing a DNA 5mer array in this embodiment includes at least one instance of every possible DNA 5mer sequence permutations on the substrate. Other examples of methods for manufacturing DNA, RNA, and nucleic acid analog oligomers will be readily apparent to those of skill in the art of manufacturing oligomer arrays upon reading this disclosure. This invention contemplates methods that include one or more nucleotide analogs instead of a DNA′ or RNA nucleotide within the oligomers provided. These methods include providing at least one oligomer example of every sequence permutation of different nucleotides and/or nucleotide analogs used in the method, for an oligomer of a particular length, where that length is anyone of a 2mer to a 15mer, inclusive. Alternatively, the method includes providing oligomers that contain only nucleotide analogs, in which case the method provides at least one oligomer example of every sequence permutation of the nucleotide analogs used in the method, for an oligomer of a particular length, where the length is any one of a 2mer to a 15mer, inclusive. Oligomers provided in these methods may contain 1, 2, 3, 4, 5 or 6 different nucleotide analogs in combination with none, 1, 2, 3, 4, 5, or 6 nucleotides.

The methods for making arrays may include covalently binding the oligomers to the substrate; for example, by covalently attaching the oligomers to a chemical (e.g. an amine or hydroxyl) group on the substrate. In a different aspect, the method for making the arrays includes non-covalently binding the oligomers to the substrate; for example binding the oligomers by electrostatic interactions. In yet a different aspect, the method for making arrays includes restricting oligomers to specific addresses by creating a physical barrier between addresses, for example, by placing oligomers within different wells on a substrate that is a microtiter plate.

In another embodiment the method for making arrays includes synthesizing the oligomers on different addresses of the substrate. In some embodiments of the method for making arrays, the oligomers are attached to the substrate by a spacer or linker molecule. In other embodiments of the method, the oligomers are not attached to the substrate by a spacer or linker molecule.

Methods of Identifying Short Oligomers that Bind Target Nucleic Acids

The central insight of this invention is the recognition that libraries and arrays of short oligomers can be used to identify short oligomers that bind target nucleic acids. Thus the invention provides a method for screening collections of short oligomers to identify short oligomers that interact with target nucleic acids.

Generally, the method for identifying a nucleic acid oligomer that binds a target nucleic acid molecule includes: providing a library or array of short nucleic acid oligomers of a particular length, where that particular length is any one of a 2mer to a 15mer, inclusive; contacting the library or array with at least one target nucleic acid that includes some tertiary or globular structure; and detecting a binding interaction between the target nucleic acid and at least one oligomer in the library or array.

In one embodiment, the interaction between the target molecule and at least one oligomer is detected quantitatively. In another embodiment, the interaction is determined qualitatively, e.g., whether there is a binding interaction or not. In some embodiments of the method, the target nucleic acid is complexed with a protein.

In another embodiment, the method includes providing an array of short oligomers of a particular length, from any one of a 2mer to a 15mer, inclusive; contacting the array with a target nucleic acid molecule, e.g. a RNA, that includes some tertiary or globular structure. In another embodiment, the method further includes detecting whether or not there is a binding interaction between the target molecule and at least one oligomer on the array, e.g., detecting qualitatively or quantitatively an interaction between the target and at least one oligomer on the array. The target nucleic acid molecule in this method may or may not be complexed with a protein.

In the most preferred embodiment, the method includes providing an array of short oligomers of a particular length, where the length is anyone of a 2mer to a 15mer, inclusive; contacting the array with a target nucleic acid molecule, e.g., a RNA, that includes some tertiary or globular structure, detecting a binding interaction between the target molecule and at least one oligomer on the array, and identifying at least one address and/or oligomer on the array, to which the target molecule has bound. In another embodiment, the method further includes entering, transferring or transmitting data representing the address and/or oligomer to which the target molecule has bound into a database.

The step of detecting an interaction between a target molecule and an oligomer can include detecting a label that is incorporated into or associated with the target molecule, e.g., detecting a radiolabel in the target molecule, detecting a fluorescent reporter molecule associated with the target molecule, or detecting a secondary label that interacts with a moiety suitable for secondary labeling that is attached to the target molecule. In another embodiment, the step of detecting an interaction between a target molecule and an oligomer can include any other technology developed for detecting a nucleic acid binding interaction with an oligomer on an array, e.g., a change in conductive resistance at an address on the array.

Any of the methods for identifying an oligomer that binds a target nucleic acid that includes a step of detecting an interaction between the oligomer and a target molecule, can optionally further include the step of washing the array under non-denaturing conditions, e.g., washing the array after the step of contacting the library or array with a target molecule and prior to the step of detecting an interaction between the target and an oligomer in the array or library. The optional washing step can include washing the substrate under non-denaturing conditions and in the presence of one or more non-target competitor molecules. For example, if the target molecule is an RNA molecule, the competitor can be a different RNA molecule, e.g., an RNA molecule that differs by fewer than 10 nucleotides from the target RNA molecule. Competitor molecules can be either unlabeled or differently labeled from the target molecule. Competitor molecules can optionally contain at least one DNA or RNA nucleotide sequence that is found or expressed in an organism. In another embodiment, the competitor molecule and the target molecule contain the same nucleotide sequence but only one of the two molecules, that is either the target or the competitor exclusively, is an RNA-protein complex.

In some embodiments of the methods for identifying an oligomer that binds a nucleic acid disclosed herein, the array includes some fraction that is a percentage between 20% and less than 100%, inclusive, of all permutations of the nucleotide bases and/or nucleotide analogs that can be assembled in an oligomer of a particular length, where that length is between a 2mer and 15mer, inclusive. The oligomers provided in these arrays may contain none 1, 2, 3, 4, 5 or 6 different nucleotide analogs in combination with none, 1, 2, 3, 4, or 5 nucleotides.

In other embodiments of the method for identifying a short oligomer that binds a nucleic acid disclosed herein, the array provides at least one example of every unique sequence order that can be assembled in an oligomer of a particular length, where that length is between a 2mer and a 15mer, inclusive, using the nucleotides and/or nucleotide analogs is present on the substrate. The oligomers provided in these arrays may contain none 1, 2, 3, 4, 5 or 6 different nucleotide analogs in combination with none, 1, 2, 3, 4, or 5 nucleotides.

The invention also provides a method for identifying an oligomer that preferentially interacts with one of two target molecules. The method includes: performing with a first target nucleic acid any one of the methods disclosed herein for identifying a short oligomer that binds to a nucleic acid with tertiary or globular structure molecule; performing the same method with a second target nucleic acid molecule; comparing the results; and identifying an oligomer that is preferentially interacts with either the first or the second target nucleic acid. In one embodiment of the method, the same method is performed on the two target molecules simultaneously. In another embodiment, the same method is performed on the two target molecules sequentially. In another embodiment of the method, one of the target molecules contains the same nucleic acid sequence as the other target sequence, but one of the two molecules is in a nucleic acid-protein complex, e.g., an RNA-protein complex. Examples of target nucleic acid pairs that can be used in this method include: RNA expressed by a pathogen and RNA expressed by a non pathogen; the genomic RNA from a virus and an RNA found in the host of the virus.

Any of the methods disclosed above for identifying an oligomer that binds, or binds preferentially, to a nucleic acid molecule is readily adaptable to a method for identifying a candidate for the treatment of an RNA virus. The methods for identifying such a candidate treatment include using a target nucleic acid molecule with at least a fraction of a viral nucleic acid sequence in any one of the methods disclosed above for identifying an oligomer that binds, or binds preferentially, to a nucleic acid molecule. Examples of viral nucleic acid sequence that can be used are at least portions of genomic strands from a RNA virus, a retrovirus, a dsRNA virus, a (+)sense RNA virus, a (−) RNA virus, a viroid, a satellite RNA, a prion encoding gene, Hepatitis C virus, Human Immunodeficiency virus, Herpes virus, Kaposi's sarcoma-associated herpesvirus, Coronavirus, Bovine Coronavirus, Bovine viral diarrhea virus, GB virus-B, GB virus-C, Classic swine fever virus, foot-and-mouth disease virus, Friend murine leukemia virus, Moloney murine leukemia virus, Rous' sarcoma virus, Harvey sarcoma virus, Rhopalosiphum padi virus, Cricket paralysis virus, poliovirus, rhinovirus, encephalomyocarditis virus, and hepatitis A virus, or Plautia stali intestine virus (PSIV). Oligomers that bind, or bind preferentially, to a viral nucleic acid sequence are oligomer candidates for a treatment of an RNA virus.

Any of the methods disclosed above for identifying an oligomer that binds, or binds preferentially, to a nucleic acid molecule is also readily adaptable to a method for identifying candidate oligomers useful in regulating gene expression. The methods for identifying a candidate gene expression regulating oligomer include using a target nucleic acid molecule that includes at least a fragment of an mRNA in any one of the methods disclosed above for identifying a short oligomer that binds a nucleic acid sequence. Examples of mRNA molecules whose fragments can be used in this method include: RNA transcripts from any of the following genes: an oncogene, a tumor suppressor gene, a cell adhesion molecule gene, a telomerase, a translation initiation factor, e.g. eIF4G or DAP5; a transcription factor e.g., c-myc, NF-B repressing factor (NRF); a growth factor e.g. Vascular endothelial growth factor (VEGF), Fibroblast growth factor 2 (FGF-2), Platelet-derived growth factor B (PDGF-B); a homeotic gene e.g. Antennapedia; a Survival Protein e.g. X-linked inhibitor of apoptosis (XIAP), Apaf-1; or BiP. Oligomers that bind, or bind preferentially, to an mRNA sequence are candidates for regulating the expression of the genes encoding the mRNA.

Methods of Characterizing Short Oligomers that Bind a Nucleic Acid Target Other embodiments of the present invention provide methods for characterizing a short oligomer that binds a target nucleic acid. These methods include performing any one or more of the methods disclosed herein to identify a short oligomer that binds a nucleic acid, then performing a method for characterizing the oligomer identified as binding a target nucleic acid. Examples of methods for characterizing the identified oligomer include: contacting the identified oligomer to the target nucleic acid in solution and then performing a gel-shift assay, a footprinting assay, or an affinity cleavage assay.

In a preferred method for characterizing an oligomer that binds a nucleic acid target, the identified oligomer is contacted with a cell, and the cell is then evaluated. Methods for evaluating a cell include any of the following: monitoring gene expression within the cell, monitoring the ability of a virus to enter, replicate, form infectious particles in the cell, and/or evaluating the cell's oncogenic or apoptotic properties.

### Pharmaceutical Compositions

Another embodiment of the present invention discloses a method for manufacturing a pharmaceutical composition. Generally, the method includes identifying an oligomer that binds a nucleic acid target using any one of methods and/or products disclosed herein, then preparing the identified oligomer in a pharmaceutical composition. Methods for preparing oligomers in pharmaceutical compositions are well known in the pharmaceutical arts, and some are exemplified herein. For example, the oligomers may be formulated into pharmaceutical compositions that include liposomes or microencapsulation materials.

In one embodiment, the method for preparing a pharmaceutical composition includes performing the method for identifying oligomer candidate treatments for an RNA virus, then preparing the identified oligomer candidate treatment in a pharmaceutical composition. In another embodiment, the method for preparing a pharmaceutical composition includes performing the method for identifying an oligomer candidate for regulating gene expression disclosed above, then preparing the identified oligomer candidate for regulating gene expression in a pharmaceutical composition.

In the methods herein, the preparation of the pharmaceutical composition can include the step of combining the identified oligomer with a pharmaceutically acceptable carrier.

Method for Identifying Candidate Therapeutic Target Sequence within an RNA Molecule

This invention provides a method for identifying a candidate therapeutic target sequence within an RNA molecule. The method includes using one of the library or arrays of short oligomers disclosed herein, contacting the library or array with a candidate RNA target molecule, detecting whether or not the candidate has bound one of the oligomers in the library or array, thereby identifying whether or not the target RNA molecule contains a target therapeutic sequence that can be bound by a short oligomer. In a preferred embodiment, the method further includes identifying the oligomer to which the target therapeutic sequence within the RNA target molecule has bound.

Method for Designing an Oligomer that Binds to a Target Nucleic Acid Molecule

This invention provides a method for designing an oligomer that binds to a target nucleic acid molecule. The method includes providing any of the libraries or arrays disclosed herein, contacting the library or array with a target nucleic acid molecule, detecting whether or not the target nucleic acid molecule binds the library or array, identifying the oligomer to which the target molecule binds, and using the structural features of the oligomer to which the target molecule binds to design structural variants that bind the target molecule. The design of a structural variant oligomer typically involves designing an oligomer that chemically differs from the identified oligomer, e.g., where the structural variant has a different sugar backbone, a different base residue at one position of the oligomer, or by converting a nucleotide oligomer such as an RNA into a nucleotide analog oligomer such as a morpholino. In another embodiment, the method includes contacting one of the arrays of short oligomers disclosed herein with a target nucleic acid molecule, detecting whether or not the target nucleic acid molecule binds the array, identifying the address on the array to which the target molecule binds, correlating the address of the array with the structural features of an oligomer located at the address, and using those structural features to design structural variants that bind the target nucleic acid molecule. In another embodiment, the method includes preparing a derivative nucleic acid based on an identified nucleic acid. For example, the derivative nucleic acid can include all the bases of the identified nucleic acid, but have an altered backbone, e.g., a backbone with reduced charge or other property, e.g., a property which increases permeability. In one embodiment, the method includes formulating the identified nucleic acid or a derivative nucleic acid in a form that facilitates transport across a cellular membrane. For example, the nucleic acid can be inserted into a liposome, attached to a protein transduction domain (e.g., the transduction domain of HIV tat protein), and so forth.

### Terms

Library: A collection of oligomers. Members of the library may be intermingled. In other embodiments, members of the library may be separated from one another, e.g., in different containers, pooled in subsets, or disposed at different addresses on a surface. Exemplary oligomers are composed of DNA, RNA, or nucleic acid analog. Array: a substrate having one or more pluralities of oligomers that are stably associated with the substrate. The substrate can be rigid or flexible. Oligomers may be covalently or non-covalently bound to the substrate. Non-covalent examples include non-specific adsorption, electrostatic binding, hydrophobic interactions, hydrogen-bonding interactions etc. Covalent examples include covalent interactions between the oligomer and a functional group present on the substrate (e.g. an —OH) or on a spacer or linking group. An array need not have any particular shape, nor any particular surface characteristics, nor any particular arrangement of addresses. In one embodiment, an array is a porous array. Arrays may be ordered arrays, or non-ordered arrays.

Substrate: any insoluble material. In one embodiment, a substrate provides the insoluble component of an array that can be glass, membrane, plastic, biological, non-biological, organic, or inorganic composition suitable for depositing or synthesizing oligonucleotides. Substrates includes films, array plates, particles, strands, gels, tubing, spheres, containers, capillaries, pads, slices, films, plates or slides. Substrates also include glass coated with poly-lysine, amino silanes or amino-reactive silanes. Context will indicate if the term “substrate” refers to a component of a chemical reaction, e.g., an enzymatic reaction, rather than an insoluble material.

Support: material attached to an oligomer, examples include magnetic beads, porous glass beads, cellulose, sepharose, streptavidin coated particle, glass, nylon, and silicon.

Address: a location on a substrate, distinguishable from other locations on the substrate. It is possible for adjacent addresses to be separated by a gap or barrier, or for addresses to be immediately adjacent so that a border is formed between them, or for addresses even to be partially overlapping so long as the addresses can be distinguished. Oligomers may be associated with an address, e.g. by being attached to the address.

Spacer: an organic or inorganic molecule attached to the substrate that covalently or non-covalently binds an oligomer, thereby indirectly attaching the oligomer to the substrate. E.g. polyethylene glycol conjugated to an aminoalkylsilane, the spacer can be a nucleic acid or a sugar.

Nucleic acid: a polymer of bases organized by a sugar-phosphate backbone. Natural nucleic acids are composed of purines, pyrimidines, carbohydrates, and phosphoric acid. Examples of natural nucleic acids include DNA and RNA.

Nucleic acid analog: an oligomer containing at least one nucleotide analog.

Nucleotide analog: a chemically modified DNA or RNA nucleotide, such that the nucleotide analog can be incorporated into a nucleic acid analog.

Oligomer: a nucleic acid polymer molecule. Typically, the oligomer is a polymer that includes are nucleotides or nucleotide analogs. It some embodiments, the oligomer is a “short oligomer.” A “short oligomer” refers to an oligomer that is less than 16 monomers in length, e.g., 15, 14, 13, 12, 11, 10, 9, 8, 7, 6, 5, 4, 3, or 2 monomers in length.

Oligomer Type: the composition of the monomers in an oligomer. Thus oligomers containing solely DNA are of a different type as oligomers containing solely RNA, and both of these are a different type of oligomer from oligomers containing a mixture of DNA and nucleotide analogs. Some oligomers, however, are a combination of different oligomer types.

Target RNA or Target DNA: a polymeric molecule of RNA or DNA, respectively, having intramolecular tertiary or globular structure. A target RNA and DNA may be referred to as a “target molecule”. The target molecule can be associated with another molecule such as a protein, a carbohydrate, a lipid or another nucleic acid. Target molecules can be synthetically produced, produced in vitro, recombinantly produced, or harvested from cells.

Tertiary or globular structure: refers to the non-linear spatial organization or structure of a molecule.

Label: refers to ah identifiable moiety that may be attached to or incorporated in a molecule and that allows the detection of the molecule to which the label is attached or incorporated.

Commonly known labels in the art are radioactive, enzymatically active, optically detectable, or spectroscopic labels.

Fluorescent label: one of many molecules that are well known in the art including the general classes of labels: chromophores, fluorophores, and chemiluminescent moieties.

Reporter molecule: molecule comprising or attached to a label.

Radiolabels: a radioactive marker attached or incorporated into a molecule. For DNA or RNA radiolabels include ³²P, ³³P, ³⁵S, ¹⁴C, ³H which can be incorporated into the phosphate sugar backbone of DNA, RNA, or nucleotide analog oligomers.

Moiety suitable for secondary labeling: a moiety that allows indirect detection of the molecule conjugated to the moiety suitable for secondary labeling.

Detector: a device that can detect the interaction between the target nucleic acid molecule and an oligomer. The Detector can vary depending on the type of library or array used. In a preferred embodiment the detector is a scanner can detect the fluorescence emission from a fluorescent label. Alternatively the detector may be a spectroscopic or another type of detector. The scanning system for an array may make use of a moving detector relative to a fixed substrate, a fixed detector with a moving substrate, a combination, or may be able to image a sufficient region of the array without any movement. Alternatively, mirrors or other apparatus can be used to transfer the signal directly to the detector. Another exemplary detector can include electronics that can detect binding between a target nucleic acid and an immobilized nucleic acid.

Viral Nucleic Acid Sequence: a sequence of nucleotide bases from the genetic material of a virus. As used in this application, molecules comprising a viral nucleic acid sequence may be produced synthetically, produced in vitro, produced recombinantly, or such molecules may be harvested from cells infected with a virus.

The full length viral sequence: refers to the entire sequence of a viral gene, a viral transcript, or viral genome. Molecules comprising a viral nucleic acid sequence may be produced synthetically, produced in vitro, recombinantly, or such molecules may be harvested from cells infected with a virus.

Fragment of a viral nucleic acid sequence: a molecule comprising less than the entire genomic sequence of a virus.

Identified Oligomer: a short oligomer that is identified using the methods of the present invention as binding a target molecule.

Regulating Gene Expression: the regulation of gene transcription and/or translation of the gene product.

### DETAILED DESCRIPTION

The present invention provides libraries and arrays of short oligomers and methods for using libraries and arrays to identify short oligomers that bind target nucleic acid molecules. We have discovered that short oligomers are remarkably useful agents for targeting other nucleic acid molecules, such as RNA. In particular, short oligomers can selectively and stably bind to a folded region of an RNA using an interaction that does not completely depend on Watson-Crick or Hoogstein base-pairing, and typically involves predominantly, interactions other than such base-pairing interactions. The energetics of these interactions differ substantially from helix-forming base-pairing so that, although these short oligomers would generally fail to form stable helices, they are effective at binding to a folded target nucleic acid. Short oligomers with these and related binding properties can be used as therapeutics, lead compounds, or reagents for detecting a target.

Because RNA molecules more readily exhibit tertiary structures, the present invention will be especially useful in the identification of short oligomers that bind target RNA molecules that contain tertiary or globular structural features.

### Libraries and Arrays

The libraries and arrays of the present invention contain a plurality of short oligomers. These oligomers in the library may be composed of RNA, DNA, a nucleic acid analog or some combination of RNA and/or DNA and/or a nucleic acid analog. The length of these short oligomers can be any length between a 2mer and a 15 met, inclusive.

Methods of manufacturing DNA, RNA, and nucleic acid oligomers for use in libraries and arrays are well known in the art. These methods include variety of chemical synthesis protocols. Apparatus for synthesizing oligomers are well known and commercially available from manufacturers such as ABI™ Norwalk, Conn. and BioAutomation™ Plano, Tex.

Oligomers in the present invention are DNA, RNA, and/or a Nucleic acid analog. By DNA is meant a deoxyribonucleic acid. RNA refers to a ribonucleic acid. Nucleic acid analog refers to any of the wide variety of molecules that are recognized by practitioners in the art as being molecules that are chemically similar to DNA or RNA, composed of chemical substituents that can be assembled into an oligomer, and which are capable of binding a nucleic acid. Examples of nucleic acid analogs include nucleotide analogs and peptide nucleic acids. Nucleotide analogs include chemically modified variants of DNA of RNA, including modifications to one or more of the following chemical structures of a DNA or RNA molecule: the base, the sugar, the internucleoside phosphate linkages, and further including molecules having added substitutents such as diamines, cholesterol, lipophilic groups. One notable DNA analog is known in the art as morpholinos. Types of modified internucleoside phosphate linkages that characterize examples of DNA and RNA nucleotide analogs include: phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate and/or sulfone internucleotide linkages, or 3′-3′, 5′-2′ or 5′-5′ linkages, and combinations of such similar so linkages (to produce mixed backbone modified oligonucleotides). Examples of additional nucleotide analogs are described in Gallo et al., Design and applications of modified oligonucleotides (2003) Brazilian Journal of Medical and Biological Research 36:143-151; Luyten I. and Herdewijn, P. (1998) Hybridization Properties of base modified oligonucleotides within the double and triple helix motif, Eur. J. of Medicinal Chemistry; Herdewijn, P. (2000) Heterocyclic modifications of oligonucleotides and antisense technology, Antisense and Nucleic Acid Drug Development, 10: 297-310; Seeberger, P H and Acaruthers K H (1997) Modified oligonucleotides as antisense therapeutics in Applied Antisense Oligonucleotide Technology Stein C A and Krieg A M, eds., Wiley-Liss Inc. New York 51-72. Freier S M and Altman K H (1997) The ups and downs of nucleic acid duplex stability: structure-stability studies on chemically modified DNA:RNA duplexes, Nucleic Acids Research, 25:4429-4443.

Peptide nucleic acids (PNAs) are a class of oligonucleotide analogs wherein the entire deoxyribose phosphate backbone has been replaced by a chemically different, structurally homomorphous backbone composed of (2-aminoethyl)glycine units. Despite this dramatic change in chemical makeup, PNAs recognize complementary DNA and RNA by Watson-Crick base pairing. Furthermore, PNAs have been shown to have numerous advantages over, DNA and is RNA oligomers. For example, PNAs lack 3′ to 5′ polarity and thus can bind in either a parallel or an antiparallel orientation to DNA or RNA (Egholm, M. et al., Nature 365:566, 1993). It has been demonstrated that PNAs can bind double-stranded DNA by invading the DNA duplex and displacing one strand to form a stable D-loop structure (Peffer et al., Proc. Natl. Acad. Sci. USA 90:10648, 1993). A further advantage of PNAs is that they are less susceptible to enzymatic degradation (Demidov et al. Biochem. Pharmacol. 48:1310, 1994) and bind RNA with higher affinity than analogous DNA oligomers (Norton et al. Nature Biotechnology 14:615, 1996).

In one embodiment libraries can be made according to a randomized DNA library strategy. (for examples see “Design Synthesis, and Amplification of DNA Pools for Construction of Combinatorial Libraries Pools and Libraries” (2000) in Current Protocols in Molecular Biology, Vol. 4, Unit 24.2, Ausubel et al., eds., John Wiley & Sons Inc., New York; Davis, P., and Ecker, D. J. (1996) in Methods in Molecular and Cellular Biology 40, Pinilla, C., and Houghton, R. A., eds., p. 23-33, John Wiley & Sons Inc., New York; Lima et al., Combinatorial Screening and Rational Optimization for Hybridization to Folded Hepatitis C Virus RNA of Oligonucleotides with Biological Antisense Activity, Journal of Biological Chemistry; 272: 626-38; 1997). A mixture of oligomers can be made on an oligonucleotide synthesizer (e.g. ABI model 394) using experimentally determined adjusted proportions of phosphoramidites of each of the four nucleotide bases (assayed by ratio of incorporation into all possible dimers) such that, when mixed into a single vial, equimolar incorporation of all four bases at each sequence position is reproducibly obtained, thus ensuring equimolar representation of all possible sequence oligonucleotides. Limited hydrolyses using snake venom phosphodiesterase I and examination of products by uv absorption on RP-HPLC can be used to confirm the equimolar representation of bases.

Alternatively, a completely randomized library composed of short oligomers may be prepared by manually or automatically directing the synthesis of each individual oligomer that is to be represented in the library. Such a strategy can be more time consuming but insures equimolar representation of every oligomer sequence. Again commercially available synthesizers may be used for this purpose.

A preferred way of building a short oligomer library uses nucleic acid array technologies. Arrays generally refer to any support that can contain a plurality of addresses suitable for the synthesis or deposition of nucleic acid or nucleic acid analogue oligomers. The support can be rigid or flexible and will contain a substrate suitable for depositing or synthesizing oligomers. The substrate can be made of glass, plastic, polymer, biological, non-biological, organic, inorganic materials suitable for depositing or synthesizing oligomers. Arrays can take the forms of multiwell plates, microtiter plates, microarray plates, particles, strands, gels, tubing, spheres, containers, capillaries, pads, slices, films, or slides. The substrate and its surface may also be chosen to provide appropriate light-absorbing characteristics. For instance, the substrate may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other suitable substrate materials will be readily apparent to those of skill in the art upon review of this disclosure.

Oligomers may be synthesized on an array using a variety of techniques known to those skilled in the art of oligomer synthesis on solid supports, e.g. the methods described in U.S. Pat. No. 5,143,854 and U.S. Pat. No. 5,510,270 and U.S. Pat. No. 5,527,681. These methods, involve activating predefined regions of a solid support and then contacting the support with a preselected monomer solution. These regions can be activated with a light source, typically shown through a mask (much in the manner of photolithography techniques used in integrated circuit fabrication). Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in PCT No. 92/10183, U.S. Pat. No. 5,384,261.

Additional methods applicable to array synthesis on a single support are described in U.S. Pat. No. 5,384,261. In these methods reagents are delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

Another method which is useful for the preparation of the immobilized arrays of single-stranded DNA molecules X of the present invention involves “pin-based synthesis.” This method, which is described in detail in U.S. Pat. No. 5,288,514, utilizes a support having a plurality of pins or other extensions. The pins are each inserted simultaneously into individual reagent containers in a tray. An array of 96 pins is commonly utilized with a 96-container tray, such as a 96-well microtitre dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously. The invention provides for the use of support(s) on which the chemical coupling steps are conducted. The support is optionally provided with a spacer, S, having active sites. In the particular case of oligonucleotides, for example, the spacer may be selected from a wide variety of molecules which can be used in organic environments associated with synthesis as well as aqueous environments associated with binding studies such as may be conducted between the nucleic acid members of the array and other molecules. These molecules include, but are not limited to, so proteins (or fragments thereof), lipids, carbohydrates, proteoglycans and nucleic acid molecules. Examples of suitable spacers are polyethyleneglycols, dicarboxylic acids, polyamines and alkylenes, substituted with, for example, methoxy and ethoxy groups. Additionally, the spacers will have an active site on the distal end. The active sites are optionally protected initially by protecting groups. Among a wide variety of protecting groups which are useful are FMOC, BOC, t-butyl esters, t-butyl ethers, and the like.

Various exemplary protecting groups are described in, for example, Atherton et al., 1989, Solid Phase Peptide Synthesis, IRL Press at Oxford University Press, New York. In some embodiments, the spacer may provide for a cleavable function by way of, for example, exposure to acid or base.

Yet another method which is useful for synthesis of compounds and arrays of the present invention involves “bead based synthesis.” A general approach for bead based synthesis is described in PCT/US93/04145 (filed Apr. 28, 1993).

For the synthesis of molecules such as oligonucleotides on beads, a large plurality of beads are suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

Patents and patent applications describing arrays of oligomers and methods for their fabrication include: U.S. Pat. Nos. 6,565,569; 6,562,569; 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,895; 5,624,711, 5,639,605; 5,658,734, 5,700,637; 5,744,305; 5,837,832; 5,843,655; 5,861,242; 5,874,974; 5,885,837; WO 93/17126; WO 95/11995; WO 95/35505; EP 742 287; and EP 799 897. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; 5,874,219; WO 95/21265; WO 96/31622; WO 97/10365; WO 97/27317; EP 373 203; and EP 785 280. References that disclose the synthesis of arrays and reagents for use with arrays include: Matteucci M. D. and Caruthers M. H., J. Am. Chem. Soc. (1981) 103:3185-3191; Beaucage S. L. and Caruthers M. H., Tetrahedron Letters, (1981) 22(20):1859-1862; Adams S. P. et al., J. Am. Chem. Soc. (1983) 105:661-663; Sproat D. S. and Brown D. M., Nucleic Acids Research, (1985) 13(8):2979-2987; Crea R. and Horn T., Nucleic Acids Research, (1980) 8(10):2331-48; Andrus A. et al., Tetrahedron Letters, (1988) 29(8):861-4; Applied Biosystems User Bulletin, Issue No. 43, Oct. 1, 1987, “Methyl phosphonamidite reagents and the synthesis and purification of methyl phosphonate analogs of DNA”; Miller P. S. et al., Nucleic Acids Research, (1983) 11:6225-6242. Schena, M., et al. (1995) Quantitative monitoring of gene expression patterns with a complementary DNA microarray. Science 20: 467-470; Okamoto, T., et al. (2000) Array fabrication with covalent attachment of DNA using Bubble Jet technology Nat. Biotechnol. 18: 438-441; Hughes, T. R., et al. (2001) Expression profiling using arrays fabricated by an ink-jet oligonucleotide synthesizer. Nat. Biotechnol. 19: 342-347; Lockhart, DJ., et al. (1996) Expression monitoring by hybridization to high-density oligonucleotide arrays. Nat. Biotechnol. 14: 1675-1680. Each of these is incorporated herein by reference as exemplary methods of construction and use of arrays of the present invention. The methods of these publications can be readily modified to produce the oligomer arrays of this invention.

For some applications it is desirable to conjugate the oligomers of the library or array to a carrier protein, e.g., a serum albumin, or a protein that has some affinity for the target molecule, e.g. an nucleic acid binding protein, e.g. an RNA binding protein, or a basic protein. This strategy can be used to identify oligomers whose binding of the target molecule is enhanced by the presence of the conjugated protein. The use of trimeric protein complexes to enhance the binding of a small protein ligand has been described, e.g., in Briesewitz, R. et al. (1999), Affinity modulation of small-molecule ligands by borrowing endogenous protein surfaces, Proc. Natl. Acad. Sci. USA, Vol. 9c, pp. 1953-1958.

### Target Molecules

One embodiment of the present invention is a library or array of short oligomers, between 2mers and 15 ms, inclusively, contacted with a target nucleic acid molecule that contains tertiary or globular structure. As used in this application, the tertiary or globular structure of a nucleic acid is the non-linear spatial organization of the nucleic acid that allows the nucleic acid to bind to a short oligomer using an interaction that includes at least one non-canonical interaction. As used in this application, non-canonical binding refers to binding forces that are independent of Watson-Crick, Hoogstein base-pairing rules.

Typically, the target RNA molecule includes at least one region with a intramolecular tertiary or globular structure. Exemplary tertiary structures include hairpins, bulges, G-quartets, non-helical structures, and structures stabilized by interactions between non-contiguous nucleotides.

Target RNA molecules include molecules that contain at least a portion of a viral genomic sequence with tertiary or globular structure. Examples of RNA virus genomes that have been reported as exhibiting tertiary or globular structure include: Hepatitis C virus, Human Immnuodeficiency virus, Herpes virus, Kaposi's sarcoma-associated herpesvirus, Coronavirus, Bovine Coronavirus, Bovine viral diarrhea virus, GB virus-B, GB virus-C, Classic swine fever virus, foot-and-mouth disease virus, Friend murine leukemia virus, Moloney murine leukemia virus, Rous' sarcoma virus, Harvey sarcoma virus, Rhopalosiphum padi virus, Cricket paralysis virus, poliovirus, rhinovirus, encephalomyocarditis virus, and hepatitis A virus, Plautia stali intestine virus (PSIV). Additional suitable target RNA molecules include sequences from at least a portion of the genome with tertiary or globular structure for any of the following RNA viruses: Retroviruses (e.g. HIV, SIV, Avian leukemia virus, Human spumavirus) double stranded RNA viruses (e.g. rotavirus, blue tongue virus, Colorado tick fever virus), (+)sense virus (e.g. Hepatitis C virus, Hepatitis E virus, Hepatitis A virus, Bovine diarrhea virus1, poliovirus, human rhinovirus A, Norwalk virus, Tobacco mosaic virus), (−)sense RNA virus (e.g. Marburg virus, Ebola virus, Measles virus, Mumps virus, Sendai virus, Human respiratory syncytial virus, Rabies virus, Influenza A, B, or C virus). The target nucleic acid can also be an mRNA from a DNA virus or an integrated virus (e.g., an integrated retrovirus), etc.

In another embodiment, the target nucleic acid includes at least a segment of an mRNA containing tertiary or globular structure, e.g., a coding regions, a 5′ non-coding region, or a 3′ non-coding region. Any mRNA can be a target nucleic acid. For example, the mRNA may be a mammalian mRNA, e.g., an mRNA encodes a nucleic acid that contributes to a neoplastic disorder, e.g., a cancer, e.g., a metastatic cancer. For example, the mRNA may encode an oncogene, a signal transduction protein, a transcription factor, or a cell adhesion molecule. In another embodiment, the mRNA may be a bacterial, plant, or fungal mRNA. For example, the mRNA may contribute to pathogenicity.

Another class of target RNAs include non-coding RNAs, e.g., non-coding RNAs that have a function, e.g., a catalytic, structural, or regulatory function. Exemplary non-coding RNAs include RNA components of telomerase, signal recognition particle, the splicesomes (e.g., the U1, U2, U3, U5, U9, etc. RNAs), ribosome (e.g., components of the SS, and 16S RNAs), guide RNAs (e.g., that participate in RNA editing), snRNAs, SsrA RNA, and so forth. Still other functional RNAs can participate in nuclear and cytoplasmic transport, and viral packaging.

Additional examples of RNA containing tertiary or globular structure include mRNAs that encode the following: translation initiation factors, e.g. eIF4G or DAP5; transcription factors e.g., c-myc, NP-B repressing factor (NRF); growth factors e.g. Vascular is endothelial growth factor (VEGF), Fibroblast growth factor 2 (FGF-2), Platelet-derived growth factor B (PDGF-B); homeotic genes e.g. Antennapedia; Survival Proteins e.g. X-linked inhibitor of apoptosis (XIAP), Apaf-1; and BiP. Other RNA molecules will be recognized as containing tertiary or globular structures, for example, mRNAs containing Internal Ribosome Entry Sites (IRES) as well as RNAs referred to as Viroids (Avocado sun-blotch viroid, potato spindle tuber viroid) or Virusoids (e.g. Barley yellow dwarf virusoid, Tobacco ringspot virusoid)

The target viral nucleic acid sequences or target mRNAs of the present invention may be produced or isolated using a variety of means known to persons skilled in the production of RNA molecules. Such means include, but are not limited to, the synthesis of RNA molecules using RNA synthetic chemistry, production of RNA by in vitro transcription, the recombinant production of RNA and harvesting of RNA from a host cell, or by harvesting RNA from cells infected with an active virus particle.

Examples of DNA targets include telomeres, e.g., G-quartet structures, slippage complexes, e.g., slippage complexes formed by a trinucleotide repeat (e.g., CAG), a DNA replication complex, a transcription complex, DNA mismatches, DNA adducts, mutagenic lesions, and so forth.

In one embodiment of the invention target nucleic acid is labeled. Such labeling facilitates detection of a target that is bound to an oligomer in a library or on an array. Examples of methods for labeling a target nucleic acid include but are not limited to: radiolabeling the target, conjugating the target to a reporter molecule or conjugating the target to molecule capable of binding to a secondary reporter molecule. In one embodiment, the target nucleic acid is prepared by transcribing a DNA template. The transcription reaction can include one or more labeled nucleotides. For example, the nucleotide can be radiolabeled or can include a moiety that can be used to attached to a label. It is also possible to label the target nucleic acid after contacting the target nucleic acid to one or more members of the library.

Radiolabels suitable for use in the present invention include radionucleotides which can be readily incorporated into a target molecule. Examples of radionucleotides include standard ³²P, ³³P and ³⁵S labeled dATP, dTTP, dCTP, dGTP, ATP, UTP, CTP or GTP which are commercially available, e.g., Perkin-Elmer® Wellesley, Mass., Amersham Biosciences® Piscataway, N.J. Radionucleotides may be presented to cells infected with a virus, whereupon replicating virus incorporates the radiolabeled into its genomic material, which may be harvested for use in the present invention. Alternatively radionucleotides may be added to in vitro transcriptions systems such as commercially available SP6, T3, and/or T7 phage polymerase systems, which are well known in the art and also available commercially from vendors such as Promega® (Riboprobe®) Madison, Wis. or Ambion® (Megascript®) Austin, Tex.

A target molecule may be labeled by conjugating the target to a reporter molecule such as a fluorescent molecule. Fluorescent molecules are well known in the art and include: chromophores, fluorophores, and chemiluminescent moieties. More specifically such molecules include green fluorescent proteins, cyanine dyes (e.g., CYA, Cy2, Cy3, Cy3.5, Cy5, Cy5.5, Cy7, and Cy7.5) coumarin and its derivatives, e.g. 7-amino-4-methylcoumarin, aminocoumarin and hydroxycoumarin, BODIPY dyes, such as BODIPY FL, cascade blue, Cascade Yellow, fluorescein and its derivatives, e.g. fluorescein isothiocyanate, Oregon green, Marina Blue, rhodamine dyes, e.g. rhodamine red, tetramethylrhodamine and rhodamine 6G, Texas Red, eosins and erythrosins, FITC, DAPI etc. Methods for conjugating reporter molecules to nucleic acids and nucleic acid analogues are well known, see for example: Hughes, I. R. et al. Expression profiling using arrays fabricated by an ink-jet oligonucleotide synthesizer. (2001) Nat Biotechnol 19(4), 342-7; Randolph, J. B., and Waggoner, A. S. Stability, specificity and fluorescence brightness of multiply-labeled fluorescent DNA probes. (1997) Nucleic Acids Res 25(14), 2923-9; Brumbaugh, J. A., et al. Continuous, online DNA sequencing using oligodeoxynucleotide primers with multiple fluorophores. (1988) Proc Natl Acad Sci USA 85(15), 5610-4; Wilkerson, D., The Scientist 12[10]:20, May 11, 1998.

The target molecule can alternatively be conjugated to a moiety suitable for secondary labeling. A moiety suitable for secondary label is a primary label that allows indirect detection of the molecule to which it is conjugated. For example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; nuclease inhibitors, enzymes such as horseradish peroxidase, alkaline phosphatases, luciferases, etc. Preferably, the moiety suitable for secondary label and the secondary label are binding partners. For example, the label may be a hapten or antigen, which will bind its binding partner. For example, suitable binding partner pair include, but are not limited to: antigens and antibodies (including fragments thereof (FAbs, etc.)); proteins and small molecules, including biotin and streptavidin; enzymes and substrates or inhibitors; other protein-protein interacting pairs; receptor-ligands; and carbohydrates and their binding partners. Preferred binding partner pairs include, but are not limited to, biotin (or imino-biotin) and streptavidin, digoxygenin and Abs, and Prolinx™ reagents. Biotinylated nucleotides are commercially available, and may be incorporated into target molecules to make them suitable for secondary labeling or identification using streptavidin conjugates, such as Dynabeads™.

In some applications the target molecule is conjugated to a protein that has some affinity for the target molecule. Alternatively, the target RNA is contacted to an oligomer library or array in the presence of a non-conjugated protein that has an affinity for the target RNA. This strategy can be used to identify oligomers whose binding of the target molecule is enhanced by the presence of the conjugated protein See, e.g., Briesewitz, R. et al. (1999), Affinity modulation of small-molecule ligands by borrowing endogenous protein surfaces Proc. Natl. Acad. Sci. USA, Vol. 96, pp. 1955-1958 (describing trimeric protein complexes to enhance the binding of a small protein ligand).

Identifying Oligomers that Bind Target Molecules

Methods for detecting a binding interaction between an oligomer and a target molecule varies depending on the type of oligomer library or array that is utilized and/or on the type of labeling that is employed. One method for identifying an oligomer that interacts with a target molecule involves immobilizing the target molecule, contacting the target molecule with oligomers, washing oligomers that do not bind the target, capturing and identifying the oligomer that bind to the target. The target molecule may be immobilized covalently or non-covalently on a column, on a plate, on a bead, or on any other material suitable for immobilizing a target nucleic acid. Once the oligomer that binds is separated from library members that do not bind, the binding oligomer is identified. For identification purposes it is preferable that the small oligomers themselves be tagged with a unique identifier, such uniquely identified oligomers may be made according to the method of U.S. Pat. No. 6,620,584. In another embodiment, mass spectroscopy can be used. Once an accurate mass is known, it is possible to determine which possible sequences are bound. Ambiguities can be resolved by re-testing oligomers individually. In still another embodiment, the oligomer includes a tag that can be used to amplify the oligomer using PCR, LCR, or other nucleic acid amplification method. The amplified oligomer can be identified, e.g., by sequencing.

Another method for identifying oligomers that bind target nucleic acid utilizes a library oligonucleotides immobilized on the substrate of a array. In this embodiment the target nucleic acid, e.g. RNA, is labeled. The labeled target RNA is incubated with the array in such a way that the RNA molecule has an opportunity to come in contact with the oligomers immobilized on the array. After an incubation period, the array may optionally be washed, then the array is analyzed to determine where on the array, if anywhere, the target RNA binds. The address where the target RNA binds is then correlated with an oligomer that is known to be located at the address where the target RNA binds, thereby identifying the oligomer that binds the target RNA.

Typically the binding and wash conditions are non-denaturing. For example, the array and the target nucleic acid are maintained at a temperature at least 3, 5, or 10 degrees below the melting temperature of the target nucleic acid, particularly, the folded structure of interest. Exemplary binding and wash conditions have a physiological ionic strength, an ionic strength of between 0.1, 0.5, 0.8, 0.9, 1.1, 1.5, or 2 fold that of phosphate buffered saline (PBS), or an ion strength less than 2×, 1×, or 0.75×SSC sodium chloride/sodium citrate as described in Current Protocols in Molecular Biology, John Wiley & Sons, N.Y. (1989), 6.3.1-6.3.6. Exemplary binding and wash conditions have a pH between 5-10.5, 6-9, 6-8.5, 6-8, or 6.5-7.5.

Methods for determining the address to which the target binds will vary depending on the array's substrate composition and also the type of label that is used. Persons of skill in the field of arrays will recognize that a large number of commercially available array scanners are suitable for use with the present invention (e.g. scanner manufacturers include Affymetrix® Santa Clara, Calif., Axon® Union City, Calif., GeneFocus Waterloo, Ontario Canada, and Packard Bioscience® San Jose, Calif.).

In some embodiments the array will not be so large as to exclude simpler methods like autoradiography which is suitable for use with radioactive or fluorescently labeled targets. The array is exposed to an X-ray film or a phosphorimager, which is developed and read or scanned to determine the address on the array to which the target molecule is bound. Scanners and systems appropriate for developing X-rays or phosphorimager screens are commercially available, e.g. from Kodak, Hewlett Packard and Molecular Dynamics. The address is then correlated to at least one oligomer, thereby identifying an oligomer that potentially binds a target nucleic acid.

Information derived from scanning the array directly or from scanning X-ray film or a phosphorimager can be stored on a database, thereby creating a database containing information that can be used to identify small oligomers that bind to a target nucleic acid. Preferably the database is an electronic database. The information in a an electronic database can be copied and/or transferred electronically to other databases. The contents of the database may also be manipulated using software which is readily available to persons in the art from manufacturers of array scanners.

Additional implementations for identifying a useful oligomer include the following. In one implementation, a target, e.g., a target nucleic acid, is contacted to addresses of an array. Interaction between the target and the addresses are detected. Quantitative or qualitative (e.g., binary—on/off, or generic—off/low/medium/high) evaluations of the interaction are made. The evaluations can be stored, e.g., in a database table that associates addresses to evaluation information or that associates address content (e.g., oligomer sequence) with evaluation information. In another example, the evaluation is stored in the form of an image, e.g., a rasterized image, e.g., from a CCD camera used to image at least a region of the array. Evaluation information is then used to identify one or more candidate oligomers that have a detectable interaction with the target. For example, it may be useful to order candidate oligomers using the qualitative information.

In one implementation, candidate oligomers are further evaluated for specificity. In one embodiment, specificity information is obtained by contacting a non-target, e.g., a non-target nucleic acid, to addresses of an array and evaluation interactions. For example, a non-target nucleic acid may be related to the target nucleic acid, but may differ in a region of interest. For example, the target nucleic acid may be a nucleic acid from a pathogen (e.g., a virus or bacteria), whereas the non-target nucleic acid may be a homologous nucleic acid of a host organism and oligomers are desired which specifically interact with the target. Specificity can be at least 0.5, 1.0, 1.2, 1.5, 2, 5, 10, 100, or 1000 fold preference. In another example, the non-target is a pool or population of nucleic acids, e.g., mRNA extracted from a cell, ribosomal RNA, and so forth. Interactions can be evaluated to determine if the candidate oligomer has a general interaction with all transcripts, or if it has heightened specificity. Other forms of testing include o e-to-one assays, e.g., a fluorescence assay that evaluates interaction between the candidate oligomer and the target or non-target. In another exemplary assay, the target and non-target are disposed on an array, and the candidate oligomer is labeled and contacted to the array. In still another exemplary assays, different RNA species are separated in a gel and blotted to a filter. The candidate oligomer is contacted to the filter and locations at which the binds to the filter are detected. Standards and controls can be used to determine the size or identify of the species with which the candidate oligomer interacts.

Designing Oligomers that Bind Target Nucleic Acids

The present invention provides a method for designing oligomers that bind a target nucleic acid. Identification of a short oligomer's sequence that binds a target nucleic acid can be used to direct the production of large quantities of that oligomer for use in pharmaceutical compositions, such as those described below. It may also be desirable to design modified oligomers based on the base sequence of the oligomer that was identified as binding the target. For example, in some diagnostic applications it is desirable to conjugate a primary label, or a moiety suitable for use as a secondary label, to an oligomer with the same base sequence as was identified as binding the target molecule. In other applications it is desirable to conjugate a therapeutically useful molecule to an oligomer that binds a specific target molecule. Examples of therapeutically useful molecules include radioisotopes, chemicals, ribozymes, and other molecules whose usefulness is enhanced by being targeted to a nucleic acid molecule.

The sequence information of an oligomer that binds a target can also be used to design an oligomer with the same base sequence but differs chemically from the oligomer identified as binding the target molecule. For example, a DNA oligomer identified as binding a target molecule can be used as the template for designing an RNA molecule that has the same base sequence as the DNA oligomer, except that any thymine residue on the DNA oligomer will be represented by uracil residue on the designed RNA oligomer. A DNA oligomer identified as binding a target molecule can be used to design a peptide nucleic acid or a phosphorothioate DNA analog oligomer that has an identical base sequence. Stated more generally a DNA, RNA or a nucleic acid analog oligomer identified as binding target nucleic acid can be used to design a DNA, RNA and/or nucleic acid oligomer that has an identical or equivalent base residue sequence but that is chemically distinct from the oligomer originally identified as binding the target molecule.

It is also possible to use one or more oligomers to form a compound that includes at least two oligomer sequences that each interact with a target (e.g., the same or different target). In one embodiment, the compound includes multimers of a single oligomer sequence that interacts with a particular target. Such a compound can be used to bring two molecules into proximity with one another. In another embodiment, the compound includes one oligomer that interacts with a first target and a second oligomer that interacts with a second target. The compound can be linear or can be branched, e.g., a dendrimer.

Characterizing Oligomers that Bind a Target Molecule

The present invention provides methods for evaluating the interaction between an oligomer and a target molecule. In one method, a short oligomer that has been identified as binding a target is further evaluated by contacting the oligomer and the target in solution and then evaluating the oligomer's ability to bind the target (e.g., to validate the interaction detected on the Methods for assaying the ability of an oligomer to bind a target will be readily apparent to one of ordinary skill in the field of binding interactions between oligomers and target molecules. Such methods include, but are not limited to performing gel shift assays, footprinting assays, affinity cleavage assays, Fluorescence Resonance Energy Transfer (FRET) experiments, surface plasmon resonance, X-ray crystallography and other methods suitable for revealing an interaction between an oligomer and the target molecule of the present invention.

In another embodiment, the oligomer is evaluated in a functional assay, e.g., an in vitro (e.g., cell-free or cell-based) functional assay or an in vivo functional assay. In an example where the target is an mRNA, a functional assay can be ability to translate the mRNA in the presence of the oligomer. The mRNA and the oligomer can be contacted to translation reagents, e.g., an translation extract. Ability of the mRNA to be translated can be evaluated, e.g., by detecting incorporation of amino acids into nascent proteins or by detecting formation of the encoded protein or fragments thereof. In an example, where the target is a catalytic RNA or a functional RNA, the functional assay can include evaluating the ability of the target to effects its function. For example, if the target is a spliceosome component, the assay can be an in vitro splicing assay to which the oligomer is added.

In some embodiments, it is possible to perform the functional assay on a library of oligomers, e.g., without first detecting binding interactions between the oligomer and the target. In one implementation, components of the assay can be contacted to an array that includes immobilized oligomers. For example, to identify an oligomer that modulates translation of an mRNA, a translation extract and the mRNA can be contacted to the array, and then the array is evaluated to identify addresses at which translation is altered (e.g., increased or decreased). For example, to identify an oligomer that modulates splicing, splicing components can be contacted to the array, and then the array is evaluated to identify addresses at which splicing is altered (e.g., increased or decreased). For example, to identify an oligomer that modulates telomerase, telomerase and a telomerase substrate can be contacted to the array, and then the array is evaluated to identify addresses at which telomere extension is altered (e.g., increased or decreased).

Another method of further evaluating an identified oligomer involves contacting the oligomer to a cell and then evaluating the cell. The identified oligomer may optionally be prepared in a lipophilic suspension before contacting a cell, e.g. suitable lipophilic solvents or vehicles can include fatty oils such as sesame oil, or synthetic fatty acid esters, such as ethyl oleate or triglycerides, or liposomes. The identified oligomer may alternatively be prepared in any one or more of the pharmaceutical compositions described below before the oligomer contacts the cell. In still other embodiments, the oligomer is delivered to the cell using a transfection reagent (e.g., LIPOFECTAMINE™), or a procedure that facilitates uptake (e.g., electroporation). Cell evaluations can very depending on the target molecule which the identified oligomer has been shown to bind. Examples of evaluations include but are not limited to assaying the expression of a gene product within the cell, assaying a physiological function that has been correlated with the target molecule to which the identified oligomer binds or assaying for infection or replication of a virus and/or assaying for expression of viral genes.

In some embodiments, it is possible to perform the functional cell-based assay on a library of oligomers, e.g., without first detecting binding interactions between the oligomer and the target. For example, cells can be contacted to an array, and then evaluated. In another example, the cells can be cultivated in separate containers (e.g., plates or wells of an array) and then contacted with members of a library of oligomers, e.g., individually or in a pool. The split-and-pool method can be used deconvolved a pool of oligomers to identify an individual oligomer that alters function in the assay. The split-and-pool method can also be used for cell-free assays and in vivo assays.

Pharmaceutical Compositions

Pharmaceutical compositions of this invention can include a short oligomer compound, e.g., an oligomer compound identified by a method or methods described herein, or a pharmaceutically acceptable salt thereof; optionally an additional agent selected from a protein that enhances the binding of a short oligomer to a target nucleic acid, an inhibitory agent (small molecule, polypeptide, antibody, etc.), an immunosuppressant, an anti-viral agent, anti-canter agent, anti-inflammatory agent, or an anti-vascular hyperproliferation compound, a compound to treat neurological disorders, and an anti-obesity compound; and any pharmaceutically acceptable carrier, adjuvant or vehicle. Alternate compositions of this invention comprise a short oligomer compound identified by a method or methods described herein or a pharmaceutically acceptable salt thereof; and a pharmaceutically acceptable carrier, adjuvant or vehicle.

In another embodiment, the composition includes a polymer whose monomer sequence is based on the sequence of monomers in an oligomer identified by a method described herein. For example, it is possible to make an oligomer with an altered backbone relative to an identified sequence, but including the same or similar bases. The altered backbone can have reduced negative charge, e.g., phosphates can be replaced by sulfur containing group, or phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate, sulfone internucleotide, and/or peptide linkages.

The term “pharmaceutically acceptable carrier or adjuvant” refers to a carrier or adjuvant that may be administered to a patient, together with a short oligomer compound identified by a method or methods described herein, and which does not destroy the pharmacological activity of the short oligomer compound and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tween or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-.beta.-cyclodextrins, or other solubilized derivatives may also be used advantageously to enhance delivery of short oligomer compounds identified by a method or methods described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions of this invention may comprise formulations utilizing liposome or microencapsulation techniques. Such techniques are known in the art.

The pharmaceutical compositions of this invention may be orally administered in any orally acceptable dosage form including, but not limited to, capsules, tablets, emulsions and aqueous suspensions, dispersions and solutions. In the case of tablets for oral use, commonly used carriers include lactose and corn starch. Lubricating agents, such as magnesium stearate, are also typically added. For oral administration in a capsule form, useful diluents include lactose and dried cornstarch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a short oligomer compound of this invention with a suitable non-irritating excipient that is solid at room temperature but liquid at rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the short oligomer compounds of this invention include, but are not limited to, mineral oil, liquid petroleum, white petroleum, propylene glycol, polyoxyethylene-polyoxypropylene compound, emulsifying wax, and water. Alternatively, the pharmaceutical composition can be formulated with a suitable lotion or cream containing the active short oligomer compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Dosage levels of between about 0.01 and about 100 mg/kg body weight per day, alternatively between about 0.5 and about 75 mg/kg body weight per day of the target inhibitory compounds described herein are useful in a monotherapy and/or in combination therapy for the prevention and treatment of target mediated disease. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.]]

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of the disease, condition or symptoms, the patient's disposition to the disease, condition or symptoms, and the judgment of the treating physician.

All references cited herein, whether in print, electronic, computer readable storage media or other form, are expressly incorporated by reference in their entirety, including but not limited to, abstracts, article, journals, publications, texts, treatises, internet web sites, databases, patents, and patent publications.

The phrase “15mer or shorter” as used in this application refers to any oligomer that has from 1 to 15, inclusive, monomers that can be nucleotides or nucleotide analogs, e.g. a 15mer, 14mer, 12mer, 11mer, 10mer, 9mer, 8mer, 7mer, 6mer, 5mer, 4mer, 3mer 2mer or 1mer.

### Example 1

### Array

A short DNA 5mer array was made to order by Combimatrix® Mukilteo, Wash. The array contained 1,000 of the 1,025 possible DNA 5mer oligomers, and provided a separate address for each oligomer.

### Target Sequence

The target nucleic acid sequence used was the RNA transcript of the internal ribosome entry site of the Hepatitis C virus, nucleotides 1-372 (HCV-IRES). The RNA was prepared using a Megascript™ T7 kit, nuclease free water and 5-(3-aminoallyl)-UTP, which were all provided by Ambion Inc. A DNA template encoding the HCV-IRES was constructed according to standard techniques and linearized. RNA synthesis reaction mixture contained the following:

| | |
|---|---|
| RNase-free water | 2.5 μL |
| ATP (75 mM) | 2.0 μL |
| GTP (75 mM) | 2.0 μL |
| CTP (75 mM) | 2.0 μL |
| UTP (75 mM) | 1.0 μL |
| 5-(3-aminoallyl)-UTP (50 mM) | 1.5 μL |
| 10X reaction buffer | 2.0 μL |
| HCV IRES DNA (~1 μg) | 5.0 μL |
| T7 RNA polymerase | 2.0 μL |

The reaction mixture was incubated at 30° C. overnight and purified using Rneasy® kit from QIAGEN, Inc. Target Sequence was then labeled using a Cy3 monoreactive dye pack from Amersham Biosciences, Inc. Dried HCV-IRES RNA was added to 20 μL of sodium carbonate (0.1 M pH 8.5) followed by 20 μL Cy3 dye solution. The reaction mixture was mixed and incubated at room temperature for 1 hour in the dark and purified using RNeasy® mini-kit.

### Hybridization of the HCV-IRES RNA to DNA 5Mers

The array was incubated with RNase free water for 30 minutes at 37° C. then washed three times with wash buffer (300 mM NaCl, 100 mM MgCl₂). Hybridization was effected by adding a solution of HCV-IRES RNA (92.7 nM in 300 mM NaCl, 100 mM MgCl2 and 100 mM Tris pH 7.4) into the hybridization chamber. The array was incubated at 4° C. overnight in the dark and rinsed three times with wash buffer.

Identification of 5Mers that Bind HCV-IRES RNA

The array was scanned at 532 nm using a GenePix® 4000B scanner from Axon Instruments, Inc. Scanner data was analyzed using the GenePix® Pro 5.0 software provided by Axon Instruments, Inc. From each array analyzed the 40 addresses exhibiting the most fluorescence were, selected. If an address corresponding to the same oligomer was selected in 3 out of 5 arrays analyzed, that oligomer was scored as likely binding ligand for the HCV-IRES RNA.

### Results

One sample experiment identified the following six 5mers as likely binding candidates for HCV-IRES. (Index number designates the oligomer's address on the array)

| Index Number | Oligomer Sequence |
|---|---|
| 486 | GGCCC |
| 508 | GGGGG |
| 520 | GGTCC |
| 553 | GTCCC |
| 853 | TTCTC |
| 842 | TTCCC |

In this experiment, different hybridizations reactions were performed. The 40 addresses that exhibited the most intense fluorescence for each hybridization reaction are shown in Table 1.

TABLE 1

| Array 1 | | Array 2 | | Array 3 | | Array 4 | | Array 5 | |
|---|---|---|---|---|---|---|---|---|---|
| Index | Oligomer | Index | Oligomer | Index | Oligomer | Index | Oligomer | Index | Oligomer |
| 338 | GACAC | 508 | GGGGG | 332 | GAATA | 347 | GACGG | 342 | GACCC |
| 342 | GACCC | 633 | TACTA | 670 | TATTC | 350 | GACTA | 344 | GACCT |
| 346 | GACGC | 636 | TACTT | 737 | TCTTA | 351 | GACTC | 346 | GACGC |
| 376 | GATCC | 664 | TATCT | 738 | TCTTC | 364 | GAGGG | 347 | GACGG |
| 421 | GCCTC | 687 | TCATC | 739 | TCTTG | 383 | GATGG | 364 | GAGGG |
| 422 | GCCTG | 703 | TCCTC | 740 | TCTTT | 399 | GCAGG | 408 | GCCAC |
| 435 | GCGGG | 704 | TCCTG | 831 | TTATC | 412 | GCCCA | 412 | GCCCA |
| 485 | GGCCA | 727 | TCTAT | 836 | TTCAA | 413 | GCCCC | 413 | GCCCC |
| 486 | GGCCC | 728 | TCTCA | 837 | TTCAC | 414 | GCCCG | 423 | GCCTT |
| 487 | GGCCG | 729 | TCTCC | 839 | TTCAT | 424 | GCGAA | 428 | GCGCA |
| 488 | GGCCT | 730 | TCTCG | 841 | TTCCA | 425 | GCGAC | 429 | GCGCC |
| 497 | GGCTT | 731 | TCTCT | 842 | TTCCC | 426 | GCGAG | 435 | GCGGG |
| 519 | GGTCA | 732 | TCTGA | 843 | TTCCG | 428 | GCGCA | 475 | GGAGG |
| 520 | GGTCC | 734 | TCTGC | 845 | TTCGA | 429 | GCGCC | 486 | GGCCC |
| 535 | GTACA | 736 | TCTGT | 850 | TTCGT | 430 | GCGCG | 487 | GGCCG |
| 536 | GTACC | 812 | TGTTC | 851 | TTCTA | 440 | GCGTG | 488 | GGCCT |
| 553 | GTCCC | 813 | TGTTG | 853 | TTCTC | 474 | GGAGC | 489 | GGCGA |
| 567 | GTGAC | 814 | TGTTT | 854 | TTCTG | 475 | GGAGG | 490 | GGCGC |
| 568 | GTGAG | 839 | TTCAT | 855 | TTCTT | 486 | GGCCC | 491 | GGCGG |
| 583 | GTGTC | 841 | TTCCA | 866 | TTGCT | 489 | GGCGA | 492 | GGCGT |
| 584 | GTGTG | 842 | TTCCC | 869 | TTGGC | 490 | GGCGC | 493 | GGCTA |
| 585 | GTGTT | 843 | TTCCG | 871 | TTGGT | 491 | GGCGG | 497 | GGCTT |
| 596 | GTTGG | 844 | TTCCT | 872 | TTGTA | 493 | GGCTA | 501 | GGGAT |
| 599 | GTTTC | 846 | TTCGC | 873 | TTGTC | 495 | GGCTC | 502 | GGGCA |
| 601 | GTTTG | 851 | TTCTA | 874 | TTGTG | 504 | GGGCG | 503 | GGGCC |
| 602 | GTTTT | 853 | TTCTC | 875 | TTGTT | 507 | GGGGC | 504 | GGGCG |
| 626 | TACCC | 854 | TTCTG | 877 | TTTAC | 508 | GGGGG | 505 | GGGCT |
| 634 | TACTC | 855 | TTCTT | 883 | TTTCA | 520 | GGTCC | 506 | GGGGA |
| 661 | TATCC | 862 | TTGCC | 884 | TTTCC | 523 | GGTGA | 507 | GGGGC |
| 695 | TCCCC | 872 | TTGTA | 885 | TTTCG | 524 | GGTGC | 508 | GGGGG |
| 763 | TGCCC | 873 | TTGTC | 886 | TTTCT | 525 | GGTGG | 509 | GGGGT |
| 774 | TGCTC | 874 | TTGTG | 888 | TTTGC | 553 | GTCCC | 513 | GGGTT |
| 786 | TGGCG | 875 | TTGTT | 889 | TTTGG | 572 | GTGCC | 519 | GGTCA |
| 787 | TGGCT | 883 | TTTCA | 890 | TTTGT | 579 | GTGGG | 520 | GGTCC |
| 790 | TGGGG | 884 | TTTCC | 891 | TTTTA | 716 | TCGGG | 521 | GGTCG |
| 792 | TGGTA | 885 | TTTCG | 892 | TTTTC | 763 | TGCCC | 523 | GGTGA |
| 793 | TGGTC | 886 | TTTCT | 893 | TTTTG | 790 | TGGGG | 529 | GGTTG |
| 794 | TGGTG | 889 | TTTGG | 894 | TTTTT | 793 | TGGTC | 530 | GGTTT |
| 842 | TTCCC | 892 | TTTTC | | | 794 | TGGTG | 553 | GTCCC |
| 853 | TTCTC | 893 | TTTTG | | | 842 | TTCCC | 599 | GTTTC |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

A number of embodiments of the invention have been described. Nevertheless, it will be understood that various modifications may be made without departing from the spirit and scope of the invention. Accordingly, other embodiments are within the scope of the following claims.

1.-143. (canceled)

144. A library of nucleic acid oligomers or nucleotide analog oligomers, wherein: each oligomer is a 15mer or shorter; each oligomer is immobilized on a support; and

145. The library of claim 144, wherein the library comprises from 50-100%, inclusive, of all permutations of four nucleotides in an oligomer of a particular length.

146. The library of claim 144, wherein the oligomer comprises a nucleotide analog in place of at least one of the four nucleotides.

147. An array comprising:
a substrate comprising a plurality of addresses, wherein each address is associated with at least one nucleic acid oligomer, or one nucleotide analog oligomer, that is a 9mer or shorter and wherein the oligomers are stably associated with the substrate.

148. The array of claim 147, wherein each address comprises at least one nucleic acid oligomer, or one nucleotide analog oligomer, that is a 5mer or shorter.

149. The array of claim 147, wherein the array provides an address for from 50-100%, inclusive, of all permutations of the nucleic acid oligomers.

150. The library of claim 144, wherein the nucleic acid oligomer comprises RNA.

151. The library of claim 144, wherein the nucleic acid oligomer comprises DNA or a nucleotide analog, wherein said nucleotide analog is optionally selected from the group consisting of a phosphodiester, a peptide nucleic acid, a phosphoramidate, a phosphorodiamidate, a phosphorothioate, a methylphosphonate, a morpholino, a tenofovir disoproxil fumarate, a hydroxyurea, tricyclo (tc)-DNA, a 2′-deoxy-2′-fluoro-D-arabinonucleic acid (FANA), a 2′deoxy-2′fluoro, a 2′deoxy-2′amine, a 2′-O-alkyl analog, a 2′(R or S)-deoxy-2′C-alkyl, a 2′-C-alkyl, Abacavir, ddI, 3TC, d4T, ddC, AZT, Emtricitabine, 2-amino-5-(2′-deoxy-beta-d-ribofuranosyl)pyridine-5′-triphosphate (d*CTP) and 5-(2′-deoxy-beta-d-ribofuranosyl)-3-methyl-2-pyridone-5′-triphosphate (d*TTP).

152. The array of claim 147, wherein the array comprises more than one plurality of addresses and at least one plurality comprises oligomers comprising DNA or a nucleotide analog and at least one other plurality comprises oligomers comprising RNA or a nucleotide analog, wherein at least one of said pluralities comprises DNA or RNA.

153. The library of claim 144, wherein the oligomer is bound or attached to the support or substrate by a spacer, or is attached directly to the support or substrate, without a spacer, wherein said spacer is optionally a nucleic acid spacer or a non-nucleic acid spacer.

154. The library of claim 144, wherein the oligomer is conjugated to a protein carrier molecule.

155. The array of claim 147, wherein the array further comprises a target nucleic acid molecule comprising a tertiary or globular structure bound to one or more selected addresses of the array.

156. The array of claim 155, wherein the target nucleic acid molecule comprises a moiety selected from the group consisting of: a label, optionally a fluorescent label or a radiolabel; a moiety suitable for secondary labeling of the target nucleic acid molecule; and an RNA molecule comprising a tertiary or globular structure, wherein said RNA molecule optionally comprises a label selected from the group consisting of a fluorescent label and a radiolabel, or is conjugated to a moiety that enables secondary labeling of the target nucleic acid molecule.

157. The array of claim 155, wherein the target nucleic acid molecule comprises the sequence of at least a fragment of a composition selected from the group consisting of a mRNA, a rRNA, a tRNA, a non-protein coding RNA, a small RNA, a miRNA, a siRNA, a ribozyme, a spliceosome, a telomerase, a signal recognition particle, an untranslated 5′ or 3′ region within 100 nucleotides of an ATG start codon, a ribosome pause site, a rare codon, an internal ribosome entry site and a viral nucleic acid sequence, wherein said viral nucleic acid sequence is optionally derived from a source selected from the group consisting of a RNA virus, a retrovirus, a dsRNA virus, a (+)sense RNA virus, a ( ) RNA virus, a viroid, a satellite RNA and a prion encoding gene, wherein optionally said source is selected from the group consisting of Hepatitis C virus, Human Immnuodeficiency virus, Herpes virus, Kaposi's sarcoma-associated herpesvirus, Coronavirus, Bovine Coronavirus, Bovine viral diarrhea virus, GB virus-B, GB virus-C, Classic swine fever virus, foot-and-mouth disease virus, Friend murine leukemia virus, Moloney murine leukemia virus, Rous' sarcoma virus, Harvey sarcoma virus, Rhopalosiphum padi virus, Cricket paralysis virus, poliovirus, rhinovirus, encephalomyocarditis virus, and hepatitis A virus and Plautia stali intestine virus (PSIV).

158. The array of claim 155, wherein the target nucleic acid molecule is at least a fragment of the RNA transcript of a gene selected from the group consisting of an oncogene, a tumor suppressor gene, a cell adhesion molecule gene and a telomerase, or is at least a fragment of an mRNA encoding a protein selected from the group consisting of a translation initiation factor, optionally selected from the group consisting of eIF4G and DAP5; a transcription factor, optionally selected from the group consisting of c-myc and NF-B repressing factor (NRF); a growth factor, optionally selected from the group consisting of Vascular endothelial growth factor (VEGF), Fibroblast growth factor 2 (FGF-2) and Platelet-derived growth factor B (PDGF-B); a homeotic gene, optionally Antennapedia; a Survival Protein, optionally selected from the group consisting of X-linked inhibitor of apoptosis (XIAP) and Apaf-1; and BiP.

159. The array of claim 155, wherein the target nucleic acid molecule is obtained by a process comprising one or more of the following: recombinant production in a host cell; in vitro production; and harvesting from infected cells or from cells that carry a gene expressing the target nucleic acid endogenously.

160. An apparatus comprising:
the library of claim 144; and
a detector suitable for detecting an interaction between a target nucleic acid molecule and an oligomer comprised by the library.

161. The apparatus of claim 160, wherein the detector is a scanner, optionally a fluorescent scanner, and/or comprises a microscope and/or generates information that allows a determination of the address or oligomer to which a target molecule has bound, wherein said apparatus optionally further comprises a digital storage device suitable for storing information generated by the scanner or microscope.

162. A method of making a substrate suitable for evaluating oligomers, said method comprising:
providing a substrate comprising a plurality of addresses;
attaching at least one nucleic acid oligomer at each address, wherein the oligomer is a 9mer or shorter; and
wherein the substrate provides an address for at least 10% of all permutations of four nucleotides at each position of an oligomer of a particular length, or
providing a substrate comprising a plurality of addresses;
attaching at least one nucleic acid oligomer at each address, wherein the oligomer is a 5mer or shorter; and
wherein the substrate provides an address for at least 10% of all permutations of four nucleotide bases at each position of a nucleic acid oligomer of a particular length.

163. The method of claim 162, wherein the oligomer is covalently or non-covalently attached to the substrate at each address, optionally wherein the oligomer is restricted to one address on the substrate and/or is synthesized at an address on the substrate.

164. The method of claim 162, wherein the substrate provides an address for each permutation of an oligomer of particular length comprising one of four nucleotide bases at each position, wherein said address is optionally a distinct address for every permutation of the nucleic acid oligomer.

165. A method of identifying at least one nucleic acid oligomer that non-canonically binds a target RNA molecule, said method comprising:
providing an array that comprises a substrate comprising a plurality of addresses, wherein each address comprises at least one nucleic acid oligomer, wherein the oligomer is from a 2mer to a 15mer, inclusive; and
contacting the array with a target molecule that comprises a folded RNA.

166. The method of claim 165, wherein the target molecule comprises a folded RNA-protein complex.

167. The method of claim 165, further comprising one or more steps selected from the group consisting of:
detecting, qualitatively or quantitatively, a binding interaction between the target RNA molecule and the array;
identifying one or more addresses of the plurality to which the target RNA molecule has bound;
entering data representing the address to which the target RNA molecule has bound into a database; and
identifying the oligomer, or a group of oligomers comprising the oligomer, to which the target RNA molecule has bound, optionally further comprising entering data representing the oligomer, or a group of oligomers comprising the oligomer, to which the target RNA molecule has bound.

168. The method of claim 167, wherein prior to detecting the binding interaction, the substrate is washed under non-denaturing conditions.

169. The method of claim 167, wherein the contacting or washing is done in the presence of at least one non-target RNA molecule that is either unlabeled or differently labeled from the target RNA, optionally wherein the non-target RNA differs from the target RNA by fewer than 10 nucleotides and/or the non-target RNA comprises at least one RNA sequence that is expressed in an organism and/or the target RNA and the non-target RNA comprise the same RNA sequence, but only one of the RNA molecules is in an RNA-protein complex.

170. The method of claim 167, wherein the data entered into a database represents the oligomer, or a group of oligomers comprising the oligomer, to which the target RNA molecule has bound.

171. A method of identifying an oligomer that preferentially interacts with one of two RNA molecules, the method comprising:
performing the method of claim 167 with a first RNA;
repeating the method performed with a second RNA or a second RNA in an RNA protein complex;
comparing the results; and
identifying an oligomer that preferentially interacts with either the first or the second RNA.

172. The method of claim 167, further comprising a step selected from the group consisting of:
formulating the oligomer identified as interacting with the target RNA in a pharmaceutical composition, optionally administering said pharmaceutical composition to a cell or organism, and
formulating the oligomer that preferentially interacts with one of the RNA molecules in a pharmaceutical composition.

173. A method of using an oligomer identified as interacting or preferentially interacting with a target RNA molecule, the method comprising a process selected from the group consisting of:
(1) performing the method of claim 167, wherein an oligomer is identified as interacting or preferentially interacting with a target RNA molecule;
contacting the identified oligomer to a cell; and
evaluating the cell; and
(2) performing the method of claim 167, wherein an oligomer is identified as interacting or preferentially interacting with a target RNA molecule; and
further evaluating the interaction between the identified oligomer and the target molecule.

174. The method of claim 173, wherein the interaction between the oligomer and the target is evaluated in solution and optionally comprises a gel shift assay, a footprinting experiment, and susceptibility of oligomer or the target molecule to affinity cleavage.

175. A method of making a pharmaceutical composition for treating an RNA virus or regulating gene expression, the method comprising:
performing the method of claim 165; and
formulating a pharmaceutical composition comprising the oligomer identified as useful in a treatment of a virus or in regulating gene expression.

176. A method of identifying a candidate therapeutic target sequence within an RNA molecule, comprising:
providing an array comprising a substrate having a plurality of addresses, each address comprising at least one nucleic acid oligomer, and wherein the oligomer is from a 2mer to a 15mer, inclusive;
contacting the array with a candidate RNA molecule; and
detecting whether the candidate RNA molecule has bound the array;
thereby identifying whether or not the RNA molecule comprises a candidate therapeutic target for a non-canonically binding oligomer.

177. The method of claim 176, further comprising identifying the oligomer to which RNA has bound.

178. A method of designing a nucleic acid oligomer that binds non-canonically to a target RNA molecule, comprising:
providing an array comprising a substrate having a plurality of addresses, each address comprising at least one nucleic acid oligomer, and wherein the oligomer is from a 2mer to a 15mer, inclusive;
contacting the array with the target RNA molecule;
detecting whether the candidate RNA molecule binds the array;
identifying the address to which the RNA molecule binds;
correlating the address to the structural features of an oligomer comprised by the address; and
using the structural features to design an oligomer that binds non-canonically to the target RNA molecule.

179. The method of claim 165, further comprising the step of synthesizing a variant of the oligomer identified as binding or preferentially binding a target molecule, optionally wherein synthesizing the variant oligomer comprises making new oligomers that differ from the identified oligomer in one of the following manners: the variant has an altered the sugar backbone relative to the identified oligomer, at least one nucleotide in the identified oligomer is replaced with a nucleotide analog, at least one DNA nucleotide in the identified oligomer is replaced with an RNA nucleotide, at least one RNA nucleotide in the identified oligomer is replaced with a DNA nucleotide, or at least one nucleotide analog in the oligomer is replaced with a nucleotide.

180. The method of claim 179, further comprising formulating the variant oligomer in a pharmaceutical composition.

181. A kit for identifying oligomers that bind non-canonically to target nucleic acid molecules comprising:
a container; and
within that container is packaged an array comprising a substrate having a plurality of addresses, each address comprising at least one nucleic acid oligomer; wherein the oligomer is from a 2mer to a 15mer, inclusive; and wherein the oligomers are stably associated with the substrate.

182. The kit of claim 181, wherein the array is packaged with instructions for using the array in a method for identifying oligomers that bind non-canonically to target nucleic acid molecules, e.g. RNA, and/or the array further provides an address for all permutations of the nucleic acid oligomer.

183. The kit of claim 181, further comprising non-denaturing reagents.

184. The library of claim 144, wherein each oligomer is a single length selected from the group consisting of a 3-mer, a 4-mer, a 5-mer, a 6-mer, a 7-mer, an 8-mer, a 9-mer, a 10-mer, an 11-mer, a 12-mer, a 13-mer a 14-mer and a 15-mer.
